(19) United States
(12) Patent Application Publication
(10) Pub. No.: US 2023/0167492 A1
(43) Pub. Date: Jun. 1, 2023

(54) MOLECULAR DETECTION VIA ASSEMBLY OF PARTICLE COMPLEXES

(71) Applicant: NanoBio Designs, LLC, Indianola, IA (US)

(72) Inventors: Derek M. Lyons, Altoona, IA (US); Ryan Richard Skaar, West Des Moines, IA (US); Aaron T. Santos, Indianola, IA (US)

(73) Assignee: NanoBio Designs, LLC, Indianola, IA (US)

(21) Appl. No.: 17/800,600

(22) PCT Filed: Feb. 24, 2021
(86) PCT No.: PCT/US2021/019361

§ 371 Date: Aug. 18, 2022

Related U.S. Application Data
(60) Provisional application No. 62/982,771, filed on Feb. 28, 2020.

(51) Int. Cl. C12Q 1/6869; C12Q 1/6825

(52) U.S. Cl. CPC C12Q 1/6869; C12Q 1/6825

(57) ABSTRACT

Certain aspects of the present disclosure relate generally to the detection of molecules, such as biomolecules, using functionalized particles, including microparticles. In one set of embodiments, a target molecule can be determined using magnetic particles and signaling particles that are able to bind to a portion of the target molecules. After allowing the magnetic and signaling particles to bind to the target molecule, e.g., to from a complex or an assembly, a magnetic field can be used to attract the magnetic particles, e.g., to a certain po sition. Determination of whether the signaling entity is present in the location, qualitatively or quantitatively, can then be used to determine the target molecule. Other embodiments are generally directed to systems for making or using such particles or assemblies, kits including these, or the like.

### RELATED APPLICATIONS

This application claims the benefit of U.S. Provisional Pat. Application Serial No. 62/982,771, filed Feb. 28, 2020, entitled “Molecular Detection Via Assembly of Particle Complexes,” by Lyons, et al., incorporated herein by reference in its entirety.

### FIELD

Certain aspects of the present disclosure relate generally to the detection of molecules, such as biomolecules, using functionalized particles, including microparticles. For example, some embodiments are directed to the detection of molecules, such as biomolecules, by assembly of a complex of particles linked to the molecule of interest. In certain cases, the particles may include magnetic particles, and/or particles that provide for colorimetric determination.

### BACKGROUND

Molecular detection using self-assembling microparticles has been previously described. These typically involve the colorimetric detection of aggregates that form in the presence of molecules of interest. Other methods use dielectric, paramagnetic, phosphorescent, or other properties. However, such aggregates can be difficult to detect, and thus improvements are needed.

### SUMMARY

Certain aspects of the present disclosure relate generally to the detection of molecules, such as biomolecules, using functionalized particles, including microparticles. The subject matter of the present disclosure involves, in some cases, interrelated products, alternative solutions to a particular problem, and/or a plurality of different uses of one or more systems and/or articles.

One aspect is generally directed to a method comprising exposing a target nucleic acid to a magnetic particle and a signaling particle, wherein the magnetic particle comprises a first nucleic acid sequence substantially complementary to a first portion of the target nucleic acid, and the signaling particle comprises a second nucleic acid sequence substantially complementary to a second portion of the target nucleic acid; allowing the magnetic particle and the signaling particle to each bind the target nucleic acid to form a complex; attracting the complex to a position using a magnetic field; and determining the complex by determining the signaling particle.

Another aspect is generally directed to a method comprising exposing a target molecule to a magnetic particle and a signaling particle, wherein the magnetic particle comprises a first recognition entity able to specifically bind a first portion of the target molecule, and the signaling particle comprises a second recognition entity able to specifically bind a second portion of the target molecule; allowing the magnetic particle and the signaling particle to each bind the target nucleic acid to form an complex; attracting the complex to a position using a magnetic field; and determining the complex by determining the signaling particle.

Yet another aspect is generally directed to an article comprising a complex of a target nucleic acid bound to a magnetic particle and to a signaling particle, wherein the magnetic particle comprises a nucleic acid sequence that is bound to a first portion of the target nucleic acid, and the signaling particle comprises a nucleic acid sequence that is bound to a second portion of the target nucleic acid.

Still another aspect is generally directed to an article, comprising a complex of a target molecule bound to a magnetic particle and to a signaling particle, wherein the magnetic particle comprises a first recognition entity specifically bound to a first portion of the target molecule, and the signaling particle comprises a second recognition entity specifically bound to a second portion of the target nucleic acid.

In addition, some aspects of the present disclosure encompass methods of making one or more of the embodiments described herein. Also, certain aspects of the present disclosure encompass methods of using one or more of the embodiments described herein.

Other advantages and novel features will become apparent from the following detailed description of various non-limiting embodiments when considered in conjunction with the accompanying figures.

### BRIEF DESCRIPTION OF THE DRAWINGS

Non-limiting embodiments will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the present disclosure. In the figures:

FIG. 1 shows a schematic of the components in accordance with one embodiment, including a sample of DNA to be measured, a magnetic particle coated in single-stranded DNA that is complementary to one region of the target sequence, and a fluorescent particle that is coated in single-stranded DNA that is complementary to a second region of the target sequence. In some cases, the components can be mixed together in solution. The complex can be assembled, for example, using temperature changes. For example, in some cases, e.g., for double-stranded DNA, heating above the melting point of double-stranded DNA target allows single-stranded nucleic acid sequences on the particles to hybridize to the DNA target, e.g., upon cooling below the melting temperature of the nucleic acid sequences.

FIGS. 2A-2B show that a magnet can be used to attract the self-assembled complexes into a plane for imaging, in one embodiment. In this figure, signaling particles are labeled with the letter F to indicate fluorescence properties (although in other embodiments, other types of signaling properties may be used). Magnetic particles are labeled with letter M to indicate magnetic properties. A complex of particles is illustrated as hybridized to a target molecule, e.g., a nucleic acid.

FIGS. 3A-3B show a fluidic device that may be used that facilitates quantification of target polynucleotide sequences, in another embodiment. For example, the fluidic device may be constructed of clear acrylic plastic, and it may incorporate features for injecting aqueous sample into the assembly reservoir. The device may also be constructed of other polymers or materials as well, e.g., glass or metal. If the target molecule is present in the sample, assembly of the microparticle complex may occur, e.g., in an assembly reservoir. In this example, an inclined plane provides a path for the magnetic particles to be displaced from the assembly reservoir into the imaging reservoir, e.g., upon application of an external magnetic field. Dimensions are in millimeters. However, it should be understood that this is by way of example only, and other dimensions, configurations, materials, etc. may be used in other embodiments.

FIGS. 4A-4B illustrate a fluidic device that facilitates assembly of the complex of particles in the assembly reservoir, in accordance with one embodiment. In this example, the signaling particles are label with the letter F to indicate fluorescence properties (although in other embodiments, other types of signaling properties may be used). Magnetic particles are labeled with letter M to indicate magnetic properties. Application of an external magnetic field as illustrated by an electromagnet can displace the magnetic particles into an imaging reservoir for detection, e.g., of the signaling particles. For example, for fluorescent particles, techniques such as optical imaging, microscopy, or the naked eye may be used. The presence of signaling particles in the imaging reservoir may be indicative of the presence of the target molecule in the sample, e.g., which may cause the assembly of a complex of signaling particles and magnetic particles.

FIG. 5 is an optical microscopy greyscale digital image captured at 400× optical magnification and 10× digital magnification, showing a complex of a 3 micrometer magnetic microparticle, a 3 micrometer fluorescent microparticle, and genomic DNA extracted from Zea Mays. See Example 1. Brightfield white light allows the uncolored magnetic microparticle to be visible as a spherical shape on the right, while 470 nm excitation light to allows the fluorescence microparticle on the left to be visible.

FIGS. 6A-6C show a series of optical microscopy images showing displacement of magnetic microparticles in the fluidic device shown in FIG. 4 from the assembly reservoir to the imaging reservoir by an external electromagnet. Each image is taken 10 seconds apart in time, going from FIG. 6A to FIG. 6C. Fluorescent particles visible in the imaging reservoir have been displaced from the assembly reservoir by being in a complex with the magnetic particles and genomic DNA extracted from Zea Mays. The 5 MP greyscale digital images have an exposure time of one second at 100× optical magnification. The 3 micrometer fluorescent microparticles are visible using excitation light bandpass filtered at 470 nm and emission light is bandpass filtered at 520 nm.

### DETAILED DESCRIPTION

Certain aspects of the present disclosure relate generally to the detection of molecules, such as biomolecules, using functionalized particles, including microparticles. In one set of embodiments, a target molecule can be determined using magnetic particles and signaling particles that are able to bind to a portion of the target molecules. After allowing the magnetic and signaling particles to bind to the target molecule, e.g., to from a complex or an assembly, a magnetic field can be used to attract the magnetic particles, e.g., to a certain position. Determination of whether the signaling entity is present in the location, qualitatively or quantitatively, can then be used to determine the target molecule. Other embodiments are generally directed to systems for making or using such particles or assemblies, kits including these, or the like.

One aspect pertains generally to the detection of molecules, such as biomolecules. In some embodiments, it pertains to the detection of target sequences of DNA or RNA, qualitatively and/or quantitatively, using a magnetic particle and a signaling particle. One or both of these particles may be a microparticle. In some cases, the signaling particle is a colorimetric particle, e.g., that can be determined, for example, based on color or fluorescence. As discussed herein, a suitable detector may be used to determine the signaling particles, for example, an optical or a fluorescent microscope, or other suitable detector.

For example, in some embodiments, a complex or an assembly of magnetic particles and signaling particles with a target molecule may be used. In the assembled complex, the magnetic particle may allow the signaling particle to be detected, and/or quantified. In some cases, relatively large particles, e.g., of greater than 0.5 micrometer in diameter, can be used. In some cases, the particles can be resolved by optical imaging. In addition, in certain embodiments, resolving individual particles may achieve a low Limit of Detection (LOD) where an individually resolved particle in an assembled complex can be indicative of a single target molecule that is present.

In some cases, one or both of the particles may be carboxyl-terminated polystyrene beads. Magnetic particles can be functionalized internally in some cases, with superparamagnetic iron oxide nanocrystals, although other embodiments are also possible, e.g., iron oxide particles. Similarly, signaling particles can be prepared using a variety of methods, and/or obtained commercially, for example quantum dots. For example, colorimetric particles can be functionalized internally with fluorescent dye, or a dye with characteristic absorbance maxima of electromagnetic radiation. In some cases, the particles are functionalized on the external surface by amide coupling between the surface carboxy moieties and a primary amine on the functionalization moiety. Other ways of functionalizing particles will be known to those of ordinary skill in the art.

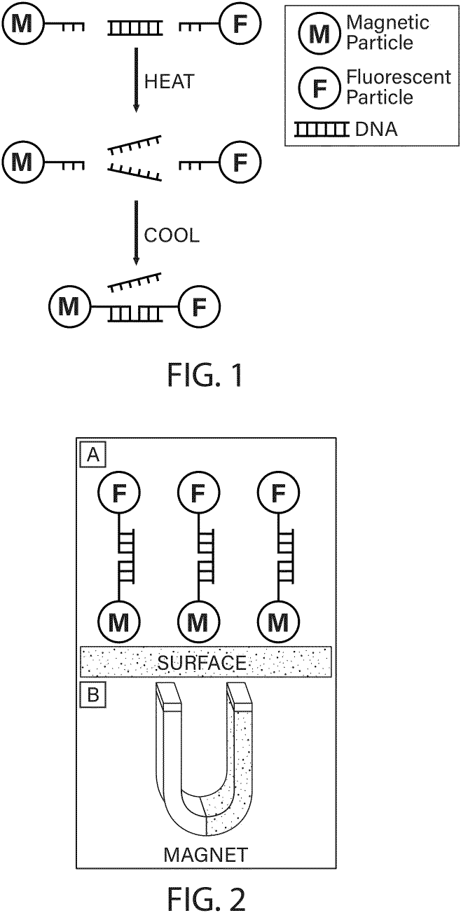

As a non-limiting example, in one embodiment, the molecules may be determined as follows. First, a nucleic acid or other suitable molecule (the “target”) is selected. For example, the nucleic acid may be a sequence of DNA. Two portions within that DNA may be identified. A set of magnetic particles may be prepared that includes nucleic acids (e.g., single-stranded DNA) that has a sequence substantially complementary to a first portion of the DNA. A set of signaling particles may also be prepared that includes nucleic acids (e.g., single-stranded DNA) that has a sequence substantially complementary to a second portion of the DNA. See, e.g., FIG. 1.

The magnetic and signaling particles may be combined together in solution along with a sample that may or may not contain the target nucleic acid sequence. For example, if the target contains double-stranded DNA, the solution may be heated to melt the DNA, i.e., separating the strands. If the sample contains DNA with the target sequence, the target DNA strands may hybridize to the substantially complementary sequences of the magnetic particles and the signaling particles, e.g., upon cooling, thereby forming a complex or an assembly. Thus, the target nucleic acid sequence may link the magnetic and signaling particles in the complex.

A magnetic field may be used to move the magnetic-fluorescent particle complex into alignment (FIG. 6), and/or to a suitable location or position. If the complex is present, it can be determined using a variety of techniques. For example, in some cases, the particles are sufficiently large that the presence of the signaling particles may be determined visually, e.g., without the need for any detectors or other equipment. In other cases, a variety of detectors may be used, for instance, microscopes, cameras, fluorescence spectrometers, or the like. The complex can be determined, for example, by measuring the amount of fluorescence observed in the imaging plane of a microscope or camera.

Such a system can be used to detect the presence or absence of target nucleic sequences within the sample, as samples that do not contain the target sequence will contain fewer signaling particles in the imaging focal plane, e.g., as the signaling particles will not be formed into assemblies along with the magnetic particles via the target nucleic acid sequences. In addition, in some embodiments, the amount of target nucleic acid can be quantified, for example, by correlating the amount of target nucleic acid in the sample to the concentration of signaling entity, e.g., as determined by fluorescence, light intensity, light scattering, etc.

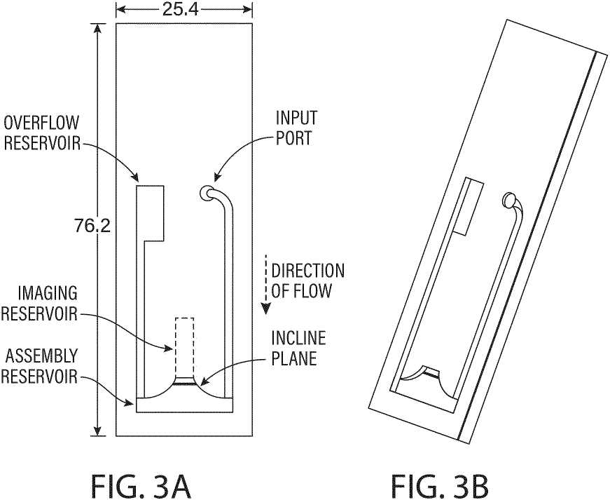

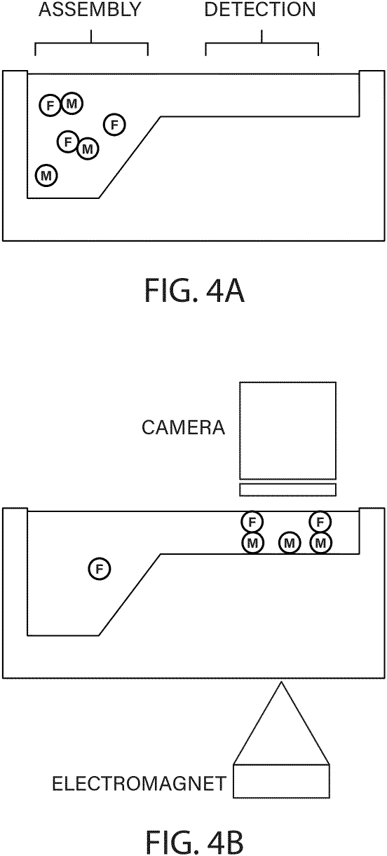

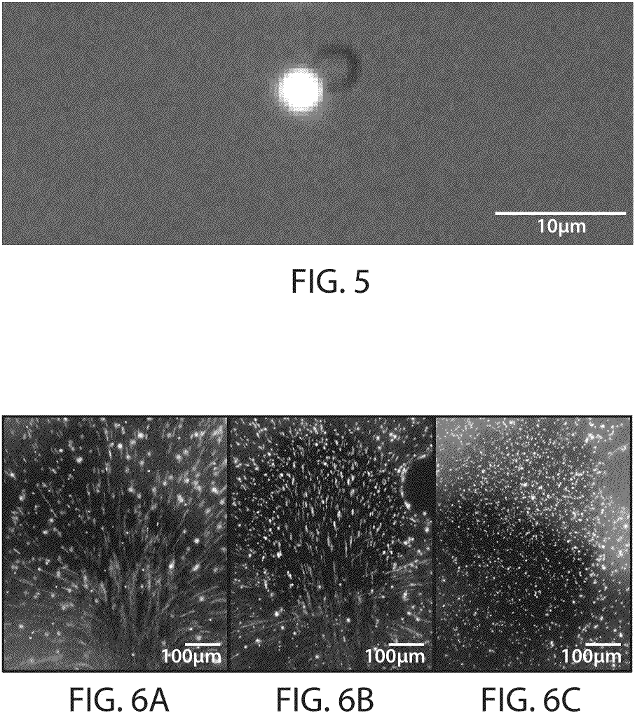

In one embodiment, a fluidic device may be used that facilitates quantification of target polynucleotide sequences. As a non-limiting example, the fluidic device may have an injection port for flowing an aqueous sample into an assembly reservoir located at the bottom of the device. A non-limiting example is shown in FIGS. 3 and 4, although other configurations are also possible. Excess sample may be allowed to flow into an overflow reservoir at the top of the device, while an overflow port allows excess sample to escape the device. A slope region connecting the assembly reservoir to the imaging reservoir allows magnetic particles, which may be assembled in a complex or assembly with the signaling particles, to be displaced from the assembly reservoir into the imaging reservoir, e.g., using an external magnetic field (see, e.g., FIG. 3). The imaging reservoir may, in some embodiments, be sufficiently shallow to aid in alignment of the assembly of magnetic and signaling particles, for example, into a monolayer (see, e.g., FIG. 4). In certain embodiments, fluid may be flowed across the monolayer to remove signaling particles, e.g., that are not in a complex or assembly.

Certain embodiments are generally directed to reducing the limitations of detecting biomolecules in biological samples. The assembly of a complex of magnetic particles and signaling particles with a target biomolecule may in some cases be used to provide quantification of the biomolecule. In one aspect, after assembly of the assembly with the target biomolecule, the magnetic particle interacts with a magnetic field to align the complex for determination, e.g., imaging, of the signaling particle.

In another aspect, after assembly of complex with the target biomolecule, an optical image of the solution containing the particles is able to resolve individual particles to provide quantification of the target biomolecule. In some cases, the number or concentration of signaling particles in the image can be correlated to the number of target biomolecules in the sample. In some embodiments, multiplexed detection of multiple biomolecular targets is accomplished by including more than one type of signaling particle, wherein each type of signaling particle is functionalized for a different biomolecular target and possesses a unique signaling property (e.g., different colors) that allows for differentiation of each particle type during imaging.

In some embodiments, the magnetic particle contains superparamagnetic iron crystals embedded in the internal structure of the particle. In some embodiments, the signaling particle has fluorescent particles or dye molecules embedded in the internal structure of the particle and/or attached to the surface of the particle. In some embodiments, the signaling particle has dye molecules embedded in the internal structure of the particle. In one embodiment, where the biomolecule target is a polynucleotide, both the magnetic particle and signaling particle may be functionalized on the surface with by a covalently attached oligonucleotide. The oligonucleotide sequence on the magnetic particle and signaling particle are complementary to two different sequences in the target polynucleotide on the same strand of the polynucleotide. Hybridization of the oligonucleotides on the particle surface to the target polynucleotide cause the complex to form. Once the complex is formed, the complex is not easily dissociated for quantification.

A variety of molecules may be determined, in various aspects. For example, in one set of embodiments, the target molecule to be determined may be a nucleic acid, for example, DNA, RNA, PNA, XNA, and/or any suitable combination of these and or other suitable polymers, and may comprise naturally-occurring bases and/or non-naturally-occurring bases. Other examples of target molecules include biomolecules, such as proteins, peptides, or the like.

In some cases, a sample suspected of containing the target molecule may be used. The sample may be, for example, cell culture fluid, water, saline, soil samples or other environmental samples, plants, agricultural samples, bacteria, fungi, blood, or another bodily fluid, such as perspiration, saliva, plasma, tears, lymph, urine, plasma, or the like. In some cases, the sample may arise from a human or any other organism, e.g., a non-human mammal. In certain embodiments, the sample arises from a pathogenic organism, e.g., a bacterium, a fungi, etc. In some cases, a sample of tissue, such as biopsy, may be taken and then homogenized or processed to separate cells. The sample, in some embodiments, may be a relatively complex or biological mixture, e.g., containing a variety of cells and/or species, and in some cases, is not well-defined.

In certain embodiments, the target molecule may be one which can be bound to a recognition entity, e.g., specifically. For example, if the target molecule is a nucleic acid, then portions of the nucleic acid may be able to bind to a substantially complementary nucleic acid sequence, e.g., of a magnetic particle or a signaling particle. As another example, portions of a molecule may be specifically recognized by antibodies, DNA-binding proteins, or the like. For instance, a magnetic particle or a signaling particle may include an antibody able to recognize the target molecule. In some cases, the binding affinity of the target molecule to the binding moiety may be less than 1 mM, less than 100 nM, less than 10 nM, or less than 1 nM.

In some cases, the target molecule may specifically bind to the recognition entity, typically to a significantly higher degree than to other molecules. For instance, the binding interaction may be at least 10×, 100×, or 1000× greater than for any other molecules that are present. In some cases, the binding may be essentially irreversible, although it need not be in other cases. Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding interactions between binding partners may be, for example, hydrogen bonds, van der Waals forces, hydrophobic interactions, covalent coupling, or the like.

Thus, as other examples besides DNA hybridization (and/or hybridization of other nucleic acids), suitable patch systems include lock and key protein interactions such as avidin-biotin or enzyme-substrate interactions, antibody-antigen pairs, covalent coupling interactions, hydrophilic/hydrophobic/fluorinated interactions, and the like. Examples of some of these are discussed herein. As noted above, DNA may be particularly useful because of its simple programmable sequence-dependent binding rules, but binding is not limited to only DNA hybridization. In addition, in some embodiments, more than one such system may be used.

As an example, in one set of embodiments, the target molecule may be a nucleic acid, to which nucleic acid sequences can hybridize to. In some cases, portions of the target nucleic acid may be substantially complementary to the nucleic acid sequences. For example, at least 5, 6, 7, 8, 9, 10, 11, 12, 13, 14, 15, or 16 nucleotides may be complementary, thereby allowing hybridization to occur. The nucleotides may be sequential, or there may be one or more non-binding nucleotides within the substantially complementary portions. In some cases, the substantially complementary portions of the target nucleic acid may be chosen to be ones that are relatively unique, e.g., which sequences do not occur in other, non-target nucleic acids that may also be present within a sample. In some cases, e.g., if the target molecule is a double-stranded nucleic acid, then temperature may be applied to cause the strands to separate, e.g., to allow hybridization to occur, e.g., with recognition entities, such as nucleic acids that are substantially complementary to at least a portion of the target nucleic acid. For instance, the temperature may be increased to above the melting point of double-stranded target, for instance, to temperatures of at least about 80° C., at least about 85° C., at least about 90° C., at least about 95° C., etc., and then cooled, e.g., to allow hybridization to occur.

Thus, in some aspects, one or more particles may be used that are able to interact with the target molecule, e.g., through the binding of recognition entities. Examples of particles include magnetic particles, signaling particles, etc., as discussed herein. In some cases, the recognition entities may be immobilized relative to the particles, e.g., attached on the surface of a particle. A variety of methods may be used to immobilize the recognition entity. For example, the particles can be functionalized on their external surfaces by amide coupling between the surface carboxy moieties and a primary amine on the functionalization moiety. Other examples include, but are not limited to, chemical or physical binding. For example, the recognition entity may be covalently bonded to the particle via a biotin-streptavidin linkage, a His-tag/Ni-NTA linkage, or the like. In another set of embodiments, the recognition entity may be physically incorporated into the particle, e.g., upon formation of the particle.

The particle is a microparticle in certain aspects. The particle may be of any of a wide variety of types. The particle may be spherical or non-spherical, and may be formed of any suitable material. In some cases, a plurality of particles is used, which have substantially the same composition and/or substantially the same average diameter. The “average diameter” of a plurality or series of particles is the arithmetic average of the average diameters of each of the particles. Those of ordinary skill in the art will be able to determine the average diameter (or other characteristic dimension) of a plurality or series of particles, for example, using laser light scattering, microscopic examination, or other known techniques. The average diameter of a single particle, in a non-spherical particle, is the diameter of a perfect sphere having the same volume as the non-spherical particle. The average diameter of a particle (and/or of a plurality or series of particles) may be, for example, less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 20 micrometers, less than about 10 micrometers, less than about 5 micrometers, less than about 3 micrometers, less than about 2 micrometers, less than 1 micrometer, less than about 0.5 micrometers, less than about 0.3 micrometers, less than about 0.2 micrometers, or less than about 0.1 micrometers in some cases. The average diameter may also be at least about 0.1 micrometer, at least about 0.2 micrometers, at least about 0.3 micrometers, at least about 0.5 micrometers, at least about 1 micrometer, at least about 2 micrometers, at least about 3 micrometers, at least about 5 micrometers, at least about 10 micrometers, at least about 20 micrometers, at least about 30 micrometers, at least about 50 micrometers, at least about 100 micrometers, at least about 200 micrometers, at least about 300 micrometers, at least about 500 micrometers, or at least about 1 mm in certain cases. In some cases combinations of these are also possible, e.g., the particle may have an average diameter of between 0.2 micrometers and 1 micrometer, between about 100 micrometers and about 200 micrometers, etc.

In some embodiments, the particles may comprise one or more polymers. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers. In some cases, the particle may be a polymer-coated particle, such as polystyrene coated gold particle, polyethylene coated silica particle etc.

In one aspect, at least some of the particles are magnetic. Examples of magnetic particles that may be used include iron oxide, magnetite, hematite, other compounds containing iron, or the like. In some cases, the particles may comprise iron crystals or particles, e.g., which may be superparamagnetic in some embodiments.

In some aspects, at least some of the particles are signaling particle, e.g., that can be determined using a suitable technique. For example, the particle may be a colorimetric particle, e.g., that can be determined optically, microscopically, using fluorescence, etc. The particle may include a signaling entity such as a dye, a fluorescent dye, a chemiluminescent entity, a radioactive label, an isotope such as a non-radioactive isotope or an isotope detectable by mass spectrometry, a ligand which can serve as a specific binding partner to a labeled antibody, an enzyme, an antibody which can serve as a specific binding partner for a labeled ligand, an antigen, a group having a specific reactivity, and/or an electrochemically detectable moieties. Non-limiting examples of dyes or fluorescent signaling entities include fluorescein, calcein, rhodamine, Green Fluorescent Protein (GFP), etc. Those of ordinary skill in the art will be aware of other fluorescent entities that are readily commercially available.

Such particles may be determined, in some cases, using a suitable detector, although in certain embodiments, the particles can be determined unaided, e.g., to the naked eye. Examples of suitable detectors include, but are not limited to, microscopes (e.g., fluorescent microscopes), plate readers, Geiger counters, mass spectrometers, or the like.

In some cases, the presence or absence of the signaling particle is determined, e.g., whether the particles exceed a certain background threshold is determined. In other embodiments, however, the concentration of the signaling particles can be determined, e.g., by determining the intensity of the signal for the signaling entity. As a non-limiting example, if the signaling particle is fluorescent, then the amount of fluorescence may be related to the concentration of target molecule that is present in the sample.

In some aspects, both signaling particles and magnetic particles may be present. These particles may independently be of the same or different sizes, and independently comprise the same or different materials. In some cases, they may both have similar recognition entities (e.g., both may have nucleic acid sequences able to recognize different portions of a nucleic acid), although in some cases, different recognition sequences may independently be used.

In one set of embodiments, a signaling particle and a magnetic particle can each recognize a target molecule, e.g., via recognition entities that may be present in both particles. If the target molecule is present, both can bind to the target, resulting in an assembly of a magnetic particle, a target, and a signaling particle. However, if the target molecule is not present, then the signaling particle and the magnetic particle are unable to come together to form an assembly. Accordingly, by adding the signaling particle and a magnetic particle to a sample, the presence and/or concentration of a target molecule may be determined, e.g., qualitatively and/or quantitatively.

In some cases, after allowing assembly to occur, a magnetic field may be applied to a sample to cause the magnetic particles to move to a particular location, e.g., within the sample. If assemblies of the magnetic particles and signaling particles are present, e.g., due to the target molecule, then at least some of the signaling particles will also be brought to the location, e.g., increasing their overall concentration. However, if no target molecule is present, then the magnetic particles will not be associated or assembled with the signaling particles, and thus, movement of the magnetic particles to particular location will not increase the concentration of signaling particles at that location. It will be understood, however, that in some cases, there may be low or background concentrations of signaling particles at a location; however, it can readily be determined whether the concentration of signaling particles at the location changes in response to the above, e.g., which may indicate that some target molecule is present within the sample.

U.S. Provisional Pat. Application Serial No. 62/982,771, filed Feb. 28, 2020, entitled “Molecular Detection Via Assembly of Particle Complexes,” by Lyons, et al., is incorporated herein by reference in its entirety.

The following examples are intended to illustrate certain embodiments of the present disclosure, but do not exemplify the full scope of the disclosure.

### Example 1

Exemplary data describes the detection of a genomic DNA target sequence in Zea Mays. Two DNA oligonucleotide sequences were designed with complementary sequences to two regions in the Cauliflower mosaic virus CaMV 35S promoter sequence, GCCTCTGCCGACAGTGGT and GAAGACGTTCCAACCACGTCTT, and functionalized to the microparticles by incorporating a primary amine and a polyethylene glycol spacer moiety on either the 5′ or 3′ terminal end of the oligonucleotide. The amine is amide coupled to the carboxy-terminated 3 micrometer diameter polystyrene beads. Magnetic beads were embedded with iron oxide nanocrystals and the colorimetric beads are embedded with fluorescent dye with an absorption maxima at 470 nm. FIG. 4 shows an optical microscopy digital image of the complex of particles assembled in the presence of genomic DNA extracted from Zea Mays that includes the CaMV 35S DNA sequence.

10 g of whole maize seeds were ground to 40 mesh and the genomic DNA extracted into a buffered saline solution containing detergents by shaking for one minute. 0.1 mL of the solution was injected into a fluidic device where the sample mixed with the pre-loaded oligonucleotide functionalized magnetic and fluorescent microparticles. The fluidic device was heated to 95° C. for 10 seconds, followed immediately by cooling the device from 60° C. to 50° C. at 2° C. per minute. An external electromagnet displaced all magnetic particles from the assembly reservoir into the imaging reservoir and a 5 MP digital image was captured using 472 nm excitation light that was bandpass filtered at 520 nm. Binarization of the greyscale image provides quantification of all fluorescent microparticles that entered the imaging reservoir as a complex with a magnetic microparticle.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
exposing a target nucleic acid to a magnetic particle and a signaling particle, wherein the magnetic particle comprises a first nucleic acid sequence substantially complementary to a first portion of the target nucleic acid, and the signaling particle comprises a second nucleic acid sequence substantially complementary to a second portion of the target nucleic acid;
allowing the magnetic particle and the signaling particle to each bind the target nucleic acid to form a complex;
attracting the complex to a position using a magnetic field; and
determining the complex by determining the signaling particle.

2. The method of claim 1, wherein the magnetic particle is microparticle.

3. The method of claim 1, wherein the signaling particle is microparticle.

4-6. (canceled)

7. The method of claim 1, wherein the signaling particle is fluorescent.

8-20. (canceled)

21. The method of claim 1, further comprising a signaling particle comprising a nucleic acid sequence that is not substantially complementary to the target nucleic acid.

22-24. (canceled)

25. The method of claim 1, wherein the signaling particle comprises a fluorescent molecule.

26. (canceled)

27. The method of claim 1, wherein the target biomolecule is DNA.

28. The method of claim 1, wherein the target biomolecule is RNA.

29-30. (canceled)

31. The method of claim 1, wherein the target nucleic acid is present in a biological sample.

32. The method of claim 1, wherein the target nucleic acid is present in an agricultural sample.

33. The method of claim 1, wherein the target nucleic acid is present in an environmental sample.

34. The method of claim 1, wherein the target nucleic acid arises from a pathogenic organism.

35. (canceled)

36. An article, comprising:
a complex of a target nucleic acid bound to a magnetic particle and to a signaling particle, wherein the magnetic particle comprises a nucleic acid sequence that is bound to a first portion of the target nucleic acid, and the signaling particle comprises a nucleic acid sequence that is bound to a second portion of the target nucleic acid.

37. The article of claim 36, wherein the magnetic particle is microparticle.

38. The article of claim 36 , wherein the signaling particle is microparticle.

39-41. (canceled)

42. The article of claim 36, wherein the signaling particle is fluorescent.

43-53. (canceled)

54. The article of claim 36, wherein the signaling particle comprises a fluorescent molecule.

55-59. (canceled)

60. The article of claim 36, wherein the complex is present in a biological sample.

61. The article of claim 36, wherein the complex is present in an agricultural sample.

62. The article of claim 36, wherein the complex is present in an environmental sample.

63. The article of claim 36, wherein the target nucleic acid arises from a pathogenic organism.

64. (canceled)

65. A method, comprising:
exposing a target molecule to a magnetic particle and a signaling particle, wherein the magnetic particle comprises a first recognition entity able to specifically bind a first portion of the target molecule, and the signaling particle comprises a second recognition entity able to specifically bind a second portion of the target molecule;
allowing the magnetic particle and the signaling particle to each bind the target nucleic acid to form an complex;
attracting the complex to a position using a magnetic field; and
determining the complex by determining the signaling particle.

66. An article, comprising:
a complex of a target molecule bound to a magnetic particle and to a signaling particle, wherein the magnetic particle comprises a first recognition entity specifically bound to a first portion of the target molecule, and the signaling particle comprises a second recognition entity specifically bound to a second portion of the target nucleic acid.


SEQUENCE LISTING (2 sequences)

SEQ ID NO: 1 - DNA, 18 bp, Artificial Sequence
  Synthetic
gcctctgccgacagtggt

SEQ ID NO: 2 - DNA, 22 bp, Artificial Sequence
  Synthetic
gaagacgttccaaccacgtctt